(19) United States
(12) Patent Application Publication
(10) Pub. No.: US 2011/0034420 A1
(43) Pub. Date: Feb. 10, 2011

(54) NOVEL CRYSTALLINE FORM B OF (2R)-(3-AMINO-2-FLUOROPROPYL)PHOSPHINIC ACID

(76) Inventor: Ida Hoyer, Lund (SE)

Correspondence Address: WHITE & CASE LLP;PATENT DEPARTMENT, 1155 AVENUE OF THE AMERICAS, NEW YORK, NY, 10036, US

(21) Appl. No.: 12/922,477

(22) PCT Filed: Dec. 18, 2008
(86) PCT No.: PCT/SE08/51492

§ 371 Date: Sep. 14, 2010

(51) Int. Cl. A61K 31/662; C07F 9/30; A61P 11/06; A61P 11/00; A61P 11/14; A61P 1/00; A61P 1/04

(52) U.S. Cl. 514/114; 562/11

(57) ABSTRACT

The present invention relates to a novel crystalline form of (2R)-(3-amino-2-fluoropropyl)phosphinic acid, the present invention is also directed to the use of the crystalline form for the treatment of gastrointestinal disorders as well as to a pharmaceutical compositions comprising the same and a process for the preparation of the crystalline form.

### FIELD OF THE INVENTION

The present invention is directed to a novel crystalline form of (2R)-(3-amino-2-fluoropropyl)phosphinic acid, the present invention is also directed to the use of the crystalline form for the treatment of gastrointestinal disorders as well as to a pharmaceutical compositions comprising the same and a process for the preparation of the crystalline form.

### BACKGROUND

A crystalline form of the compound (2R)-(3-amino-2-fluoropropyl)phosphinic acid is described as Example 5 in EP-B1 1240172. It is prepared by reacting ammonium is hypophosphite with tent-butyl (2R)-2-fluoro-3-iodopropyl carbamate in the presence of N,O-bis-(trimethylsilyl)acetamide.

### DESCRIPTION OF THE INVENTION

An aspect of the present invention is to provide (2R)-(3-amino-2-fluoropropyl)phosphinic acid in crystalline form.

One aspect of the present invention is (2R)-(3-amino-2-fluoropropyl)phosphinic acid form B.

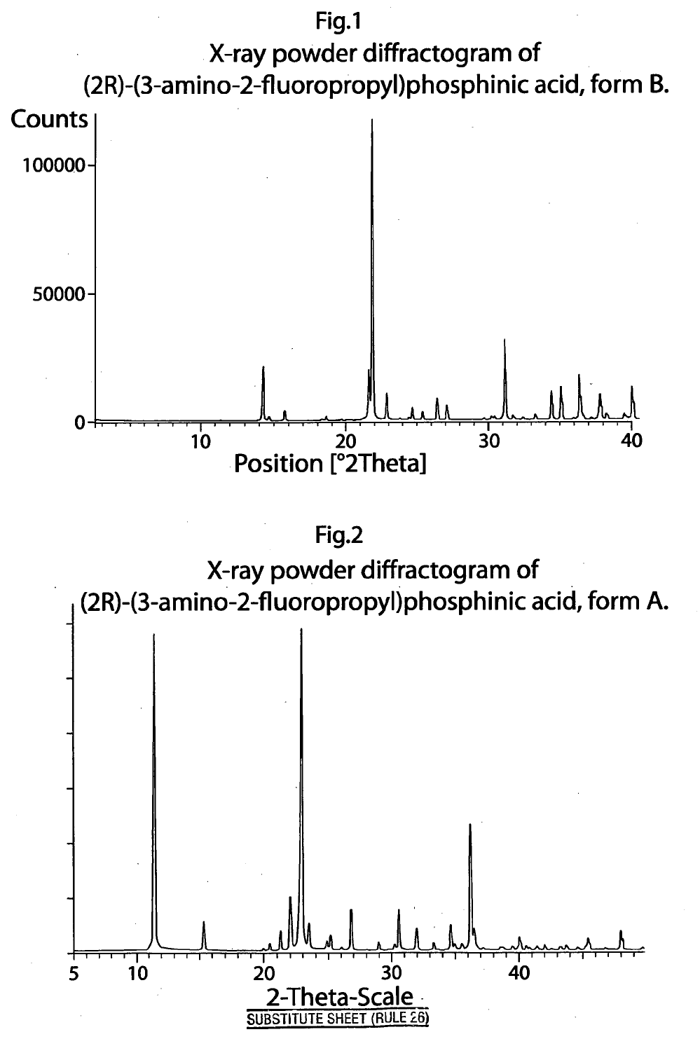

FIG. 1 is an X-ray powder diffractogram of (2R)-(3-amino-2-fluoropropyl)phosphinic acid in a crystalline form, hereinafter referred to as (2R)-(3-amino-2-fluoropropyl)phosphinic acid form B.

(2R)-(3-amino-2-fluoropropyl)phosphinic acid form B is characterized in providing an X-ray powder diffraction (XRPD) pattern, exhibiting substantially the following peaks with d-values (d-value: the spacing between successive parallel hkl planes in a crystal lattice).

Form B

| | Relative |
|---|---|
| d-value (Å) | Intensity |
| 7.7 | vw |
| 6.2 | s |
| 6.0 | w |
| 5.6 | w |
| 4.83 | vw |
| 4.75 | vw |
| 4.10 | s |
| 4.05 | vs |
| 3.88 | m |
| 3.73 | vw |
| 3.63 | w |
| 3.59 | m |
| 3.36 | m |
| 3.28 | m |
| 3.09 | vw |
| 3.00 | vw |
| 2.96 | w |
| 2.93 | w |
| 2.87 | s |
| 2.82 | w |
| 2.76 | w |
| 2.69 | w |
| 2.60 | s |
| 2.47 | s |
| 2.35 | w |
| 2.28 | w |
| 2.25 | s |

The relative intensities are presented by the following definitions:

| Definitions used | % Relative Intensity |
|---|---|
| vs (very strong): | 31-100 |
| s (strong): | 8.1-31 |
| m (medium): | 3.1-8.1 |
| w (weak): | 0.7-3.1 |
| vw (very weak): | 0-0.7 |

The relative intensities are derived from diffractograms measured with variable slits.

One embodiment of the present invention is (2R)-(3-amino-2-fluoropropyl)phosphinic acid form B, characterized in providing an X-ray powder diffraction pattern exhibiting substantially the following peak with d-value: 2.87 Å.

One embodiment of the present invention is (2R)-(3-amino-2-fluoropropyl)phosphinic acid form B, characterized in providing an X-ray powder diffraction pattern exhibiting substantially the following peaks with d-values: 6.2 Å, 4.05 Å and 2.87 Å.

A further embodiment of the present invention is (2R)-(3-amino-2-fluoropropyl)phosphinic acid form B, characterized in providing an X-ray powder diffraction pattern exhibiting substantially the following peaks with d-values: 6.2 Å, 4.10 Å, 4.05 Å, 2.87 Å, 2.60 Å and 2.47 Å.

A further embodiment of the present invention is (2R)-(3-amino-2-fluoropropyl)phosphinic acid form B, characterized in providing an X-ray powder diffraction pattern exhibiting substantially the following peaks with d-values: 6.2 Å, 4.10 Å, 4.05 Å, 3.88 Å, 3.36 Å, 3.28 Å, 2.87 Å, 2.60 Å, 2.47 Å and 2.25 Å.

The peaks, identified with d-values calculated from the Bragg formula and intensities, have been extracted from the diffractogram of (2R)-(3-amino-2-fluoropropyl)phosphinic acid form B. Additional peaks can be extracted, using conventional methods, from the diffractogram. The presence of these peaks is sufficient to establish the presence of said different polymorphs of crystalline (2R)-(3-amino-2-fluoropropyl)phosphinic acid. Merely loss of a peak does not mean that another crystalline form of the compound has been obtained.

(2R)-(3-amino-2-fluoropropyl)phosphinic acid form B is further characterized by an X-ray powder diffraction pattern essentially as shown in FIG. 1.

(2R)-(3-amino-2-fluoropropyl)phosphinic acid form B, i.e. the compound of the present invention, may be crystallized in one single solvent or in a mixture of solvents.

Crystallization may be initiated or effected with or without seeding with crystals of the compound of the invention.

In one embodiment of the present invention (2R)-(3-amino-2-fluoropropyl)phosphinic acid form B is obtained upon crystallization from a mixture of methanol, water and acetone.

A further embodiment of the present invention is to provide a process for the preparation of (2R)-(3-amino-2-fluoropropyl)phosphinic acid form B.

(2R)-(3-amino-2-fluoropropyl)phosphinic acid form B according to the present invention is substantially free from other crystal and non-crystal forms of (2R)-(3-amino-2-fluoropropyl)phosphinic acid. The term “substantially free from other crystal and non-crystal forms of (2R)-(3-amino-2-fluoropropyl)phosphinic acid” shall be understood to mean that the desired crystal form of (2R)-(3-amino-2-fluoropropyl)phosphinic acid form B contains less than 15%, such as less than 10%, or less than 5% of any other forms of (2R)-(3-amino-2-fluoropropyl)phosphinic acid.

The compound according to the present invention is useful for the inhibition of transient lower esophageal sphincter relaxations (TLESRs) and thus for treatment of gastro-esophageal reflux disorder (GERD).

The lower esophageal sphincter (LES) is prone to relaxing intermittently. As a consequence, fluid from the stomach can pass into the esophagus since the mechanical barrier is temporarily lost at such times, an event hereinafter referred to as “reflux”.

Gastro-esophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD.

One aspect of the present invention is (2R)-(3-amino-2-fluoropropyl)phosphinic acid form B for use in therapy.

The compound according to the present invention may be useful for the prevention or treatment of respiratory, cardiovascular, CNS, pain and/or gastrointestinal disorders. Examples of such disorders are asthma, such as reflux-related asthma, pulmonary diseases, cough, laryngitis, chronic laryngitis, pain, failure to thrive, gastric emptying disorders, irritable bowel syndrome (IBS), functional gastrointestinal disorder (FGD), inhibition of transient lower esophageal sphincter relaxations (TLESR), emesis, gastric motility disorders, prevention of regurgitation, belching, hiccups, functional dyspepsia, gastro-esophageal reflux disease (GERD), pharyngitis, sinusitis, otitis media, dental erosions, aspiration prophylaxis, Barett's esophageus or non-erosive reflux disease (NERD).

An aspect of the invention is the use of a compound according to the invention in a subject on patient who is a partial responder or non-responder to proton pump inhibitor (PPI) therapy.

A further aspect of the invention is a pharmaceutical composition comprising the compound according to the present invention, in admixture with a pharmaceutically acceptable carrier, diluent or excipient and optionally additional active pharmaceutical ingredients. The pharmaceutical composition may be administered in standard manner for the disease condition desired to treat, for example by oral, topical, parenteral, buccal, nasal, vaginal or rectal administration or by inhalation or insufflation. For these purposes the compound according to the present invention may be formulated into the form of, for example, tablets, pellets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

The wording “patient or subject being treated by or subjected to PPI treatment” as used herein is defined as a subject or patient that already is being treated with a proton pump inhibitor. Examples of such proton pump inhibitors are pyridinylmethylsulfinyl benzimidazoles such as omeprazole, esomeprazole, lansoprazole, pantoprazole, rabeprazole or related substances such as leminoprazole.

A further aspect of the present invention, is therapy in a patient already being subjected to treatment with an acid inhibiting agent. The wording “acid inhibiting agent” used in accordance with the present invention comprises inter alia H2 blocking agents, such as cimetidine, ranitidine, famotidine, and nizatidine.

Yet an aspect of the present invention, is therapy in a patient already being subjected to treatment with potassium channel competitive acid blockers (PCAB).

A daily dose of (3-amino-2-fluoropropyl)phosphinic acid, (2R)-(3-amino-2-fluoropropyl)phosphinic acid, or (2S)-(3-amino-2-fluoropropyl)phosphinic acid or a salt of any one of said compounds useful in a combination according to the invention, may be in the range of from 1 μg to 100 mg per day and kg body weight, such as from 10 μg to 20 mg per day and kg body weight.

In a further aspect of the invention a daily dose of (3-amino-2-fluoropropyl)phosphinic acid, (2R)-(3-amino-2-fluoropropyl)phosphinic acid, or (2S)-(3-amino-2-fluoropropyl)phosphinic acid or a salt of any one of said compounds as used herein, is up to and inclusive 10 mg/kg body weight.

In one aspect of the invention, (2R)-(3-amino-2-fluoropropyl)phosphinic acid is administered in a dosage of 65 mg b.i.d. (i.e. twice daily) as add-on therapy to omeprazole or esomeprazole or a salt of any one of said compounds, such as omeprazole or esomeprazole, or a salt of any one of said compounds.

In the practice of the invention, the most suitable route of administration as well as the therapeutic dose will depend on the nature and severity of the disease to be treated. The dose, and dose frequency, may also vary according to the age, body weight and response of the individual patient.

The compound according to the present invention may be further processed before formulation into a suitable pharmaceutical formulation. For example, the compound may be milled or ground into smaller particles before being formulated into a suitable pharmaceutical formulation.

For the avoidance of doubt, “treatment” includes the therapeutic treatment, as well as the prophylaxis, of a condition.

The presence of other substances in a sample, such as excipients, to be characterised by X-ray powder diffraction can mask some of the peaks in the above characterized crystal modification. This fact alone can not demonstrate that the crystal modification is not present in the sample. Under such circumstances due care must be used and the presence of substantially all main peaks in the X-ray powder diffraction pattern might suffice to characterize the crystal modification.

According to a further aspect of the invention there is provided a method of treatment of a medical condition, which method includes administering a therapeutically effective amount of the compound according to the present invention to a patient in need of such treatment.

### Methods of Preparation

(2R)-(3-amino-2-fluoropropyl)phosphinic acid form B may be prepared by dissolving of (2R)-(3-amino-2-fluoropropyl)phosphinic acid form A in a polar solvent, for example methanol or water or a mixture thereof. The mixture is heated to an elevated temperature, for example 40-50° C. An anti-solvent, such as acetonitrile, acetone, ethanol, isopropanol or ethyl acetate, is added over a period of hours, for example 10 hours. The mixture is stirred and held at an elevated temperature, for example at a temperature of 40° C. over a period of hours, for example at a time of 33 hours. The slurry is cooled and formed crystals are isolated and dried.

Another object is to process for preparing (2R)-(3-amino-2-fluoropropyl)phosphinic acid form B as defined above, comprising the step of treating (2R)-(3-amino-2-fluoropropyl)phosphinic acid form A in a water-containing vapor phase optionally including vapor of other solvents a period of time, wherein the relative humidity is equal to or less than 55%.

In another embodiment, (2R)-(3-amino-2-fluoropropyl)phosphinic acid form B is kept at saturated NaBr salt solution in water.

In another embodiment, the relative humidity is 51%.

In another embodiment, the temperature is 60° C.

In another embodiment, the period of time is 12 hours.

Another object is to provide a process for preparing (2R)-(3-amino-2-fluoropropyl)phosphinic acid form B as defined above, comprising the step of keeping (2R)-(3-amino-2-fluoropropyl)phosphinic acid form A as a suspension in a solvent or a mixture of solvents with a water activity equal to or less than 25%; and then isolating the product from the suspension.

In another embodiment, said solvent is a mixture of ethanol (90%, vol./vol.) and water (10%, vol./vol.).

In another embodiment, said solvent is a mixture of acetone (50%, vol./vol.), methanol (40%, vol./vol.) and water (10%, vol./vol.).

In another embodiment, the temperature is kept at between 5° C. and 70° C.

In another embodiment, the temperature is kept at between 20° C. and 50° C.

(2R)-(3-amino-2-fluoropropyl)phosphinic acid form A may be prepared by dissolving of (2R)-3-[(tent-butoxycarbonyl)amino]-2-fluoropropylphosphinic acid ammonium salt in a polar solvent, for example methanol, isopropanol or water and treatment of the solution with an acid at an elevated temperature, for example at a temperature of from 50-60° C. The reaction mixture is cooled to 30° C. and pH is adjusted to 5-6 by addition of a base. Inorganic salts may form which are precipitated and removed.

Crystallisation of (2R)-(3-amino-2-fluoropropyl)phosphinic acid form A may be initiated by adding an anti-solvent or a mixture of anti-solvents, for example acetonitrile, acetone, ethanol, isopropanol or ethyl acetate at an elevated temperature, for example at a temperature of from 40-70° C. The slurry is cooled and formed crystals are isolated and dried.

(2R)-(3-amino-2-fluoropropyl)phosphinic acid is a zwitterion, that may be crystallised at the isoelectric point, in this case approximately at a pH of 5.3.

As the reaction is performed during acidic conditions, the protonated species of (2R)-(3-amino-2-fluoropropyl)phosphinic acid is formed. After completed reaction the pH is adjusted to 5-6 by addition of a base in order to isolate (2R)-(3-amino-2-fluoropropyl)phosphinic acid crude as the zwitterion. The solution of (2R)-(3-amino-2-fluoropropyl)phosphinic acid works as a buffer solution and the amount of base added to reach the set pH-interval (5-6) can be varied in the range of from 1.8-2.8 equivalents.

A solute is crystallized from a primary solvent by the addition of a second solvent “anti-solvent” in which the solute is relatively insoluble. The anti-solvent is miscible with the primary solvent and brings about a solubility decrease of the solute in the resulting binary solvent mixture (see e.g. Allan S. Myerson, Handbook of Industrial Crystallization, second edition).

Bases useful for pH adjustment is for example NH₃ in methanol or ammonium acetate dissolved in methanol.

The formed crystals of (2R)-(3-amino-2-fluoropropyl)phosphinic acid form A may be recrystallised by dissolution in a polar solvent or a mixture of polar solvents such as methanol, isopropanol or water or a mixture thereof. The solution is clear filtered and the filter is washed with the polar solvent used. The temperature is kept at room temperature, and an anti-solvent or a mixture of anti-solvents, for example acetonitrile, acetone, ethanol, isopropanol, ethyl acetate or a mixture thereof are added during a period of 2 to 5 hours. The slurry is then stirred 5 to 12 hours. The formed product is filtered off and washed with the used anti-solvent and dried in vacuum.

The invention is illustrated, but not limited, by the following examples.

### EXAMPLES

### General Methods

¹H-NMR was performed on a Brucker 400 MHz spectrometer with D₂O as reference.

X-ray powder diffraction analysis (XRPD) was performed on samples prepared according to standard methods, for example those described in Giacovazzo, C. et al (1995), Fundamentals of Crystallography, Oxford University Press; Jenkins, R. and Snyder, R. L. (1996), Introduction to X-Ray Powder Diffractometry, John Wiley & Sons, New York; Bunn, C. W. (1948), Chemical Crystallography, Clarendon Press, London; or Klug, H. P. & Alexander, L. E. (1974), X-ray Diffraction Procedures, John Wiley and Sons, New York. X-ray diffraction analyses were performed using a PANalytical X'Pert Pro MPD for 96 minutes from 1 to 60° 2θ with CuKα radiation. Calculation into d-values (distance values) was done and they may vary in the range ±2 on the last given decimal place.

It will be appreciated by a skilled person in the art that XRPD intensities may vary when measured for essentially the same crystalline form, for example, preferred orientation.

As used herein,

Tᵢ refers to inner temperature,

Tⱼ refers to jacket temperature.

### Example 1

### Preparation of (2R)-(3-amino-2-fluoropropyl)phosphinic acid form B

40 g of (2R)-3-amino-2-fluoropropyl)phosphinic acid form A was added to 150 mL methanol and 65 mL water. The slurry was heated to 40° C. until all was dissolved. 320 mL of acetone was added to the solution over 10 hours. The slurry was stirred at 40° C. for 33 hours. The obtained crystals were filtered and dried in vacuum at 40° C. overnight. 36.67 g of (2R)-3-amino-2-fluoro-propyl)phosphinic acid form B was obtained after drying.

¹H-NMR (400 MHz, D₂O): δ 1.95 (1H, m), 2.15 (1H, m), 3.33 (2H, m), 5.16 (1H, dm, J=50 Hz), 7.08 (1H, d, J=528 Hz).

The crystals were analysed by X-ray powder diffraction (XRPD), see FIG. 1. The diffractogram of form B shows the following d-values given in Ångström and relative intensities:

Form B

| | Relative |
|---|---|
| d-value (Å) | Intensity |
| 7.7 | vw |
| 6.2 | s |
| 6.0 | w |
| 5.6 | w |
| 4.83 | vw |
| 4.75 | vw |
| 4.10 | s |
| 4.05 | vs |
| 3.88 | m |
| 3.73 | vw |
| 3.63 | w |
| 3.59 | m |
| 3.36 | m |
| 3.28 | m |
| 3.09 | vw |
| 3.00 | vw |
| 2.96 | w |
| 2.93 | w |
| 2.87 | s |
| 2.82 | w |
| 2.76 | w |
| 2.69 | w |
| 2.60 | s |
| 2.47 | s |
| 2.35 | w |
| 2.28 | w |
| 2.25 | s |

The relative intensities are presented by the following definitions:

| Definitions used | % Relative Intensity |
|---|---|
| vs (very strong): | 31-100 |
| s (strong): | 8.1-31 |
| m (medium): | 3.1-8.1 |
| w (weak): | 0.7-3.1 |
| vw (very weak): | 0-0.7 |

The relative intensities are derived from diffractograms measured with variable slits.

It is also possible to convert the metastable crystalline form A to form B by treatment with a water-containing vapor phase optionally including vapor of solvents like e.g. ethanol, isopropanol, acetone and the like in a controlled fashion. This transformation process is generally carried out at a temperature between 10° C. and 70° C., e g between 20° C. and 60° C. and, due to the speed of the transformation, e g between 40° C. and 60° C. The relative humidity (RH) at which the transformation is carried out is chosen so that the phase transition occurs at a reasonable speed i.e. within days rather than months, mainly above 30% RH, e g above 40% RH, but equal to or less than 55% RH. The time used is considerably influenced by the batch size, relative humidity and packing etc. and e g be from minutes to days. Typical values of temperature/relative humidity combinations for the formation of form B are (temperature (° C.\% RH)) 40° C./53% RH, 60° C./43% RH and 60° C./51% RH. The time of treatment is dependent on the conditions selected. Too high relative humidity (RH>75%) at higher temperature (e.g. about 60° C.) will dissolve the water soluble (2R)-(3-amino-2-fluoropropyl)phosphinic acid.

The transformation may also occur in a liquid phase as suspensions using solvents (water ethanol, isopropanol, acetone and the like) and mixtures thereof e.g. a mixture of ethanol (90%, vol./vol.) and water (10%, vol./vol.) or acetone (50%, vol./vol.), methanol (40%, vol./vol.) and water (10%, vol./vol.), which corresponds to a water activity of approximately 25% as calculated using Aspen Properties 12.1 (Aspen Technologies Inc., Cambridge, Mass.) at temperatures between 5° C. to 70° C. (upper limit by the boiling point of the solvent), e g from 20° C. to 50° C. The water activity must be equal to or below a water activity corresponding to 25% RH.

### Example 2

### Preparation of (2R)-(3-amino-2-fluoropropyl)phosphinic acid form B

1 g of crystalline (2R)-(3-amino-2-fluoropropyl)phosphinic acid form A was kept at 60° C. and 51% RH (saturated NaBr salt solution in water) over night and the transformation was confirmed by X-ray powder diffraction (XRPD) being identical to the diffractogram obtained in Example 1.

### Example 3

### Preparation of (2R)-(3-amino-2-fluoropropyl)phosphinic acid form A

320 g (1.11 moles) (2R)-3-[(tent-butoxycarbonyl)amino]-2-fluoropropylphosphinic acid ammonium salt dissolved in methanol (960 ml, 23.72 moles) was treated with sulphuric acid (105.43 ml, 1.90 moles) at 55° C. After complete reaction, the reaction mixture was cooled to 30° C. and pH was adjusted to approximately 5 by addition of ammonium acetate dissolved in methanol (180 g, 2.34 moles, 420 ml methanol). During the pH-adjustment ammonium sulphate and remaining ammonium acetate and other salts precipitated. The neutralised reaction mixture was clear filtrated. Isopropanol (3.84 L, 50.23 moles) was added at 50° C. and (2R)-(3-amino-2-fluoropropyl)phosphinic acid form A crystallised. The is slurry was cooled to 0° C. The crystals were isolated and dried under vacuum.

¹H-NMR (400 MHz, D₂O): δ 1.93 (1H, m), 2.13 (1H, m), 3.31 (2H, m), 5.14 (1H, dm, J=50 Hz), 7.07 (1H, d, J=528 Hz).

### Example 4

### Recrystallisation of (2R)-(3-amino-2-fluoropropyl)phosphinic acid form A

To 63.90 g (0.4248 moles) of (2R)-(3-amino-2-fluoropropyl)phosphinic acid (crude form A) was added to a mixture of 149 mL of methanol (2.48 rel vol) and 90 mL of water (1.5 rel vol). The reaction mixture was heated to 35° C. A clear solution was obtained. The solution was clear filtered and the filter was washed with methanol (61 ml, 1.02 rel vol). After filtration the solution was cooled to 25° C. The temperature was then kept at 25° C., and 480 mL (8 rel vol) of acetone was added over 3 hours and 20 minutes. The slurry was stirred at 25° C. for 5 hr before filtration. The product was filtered off and washed with acetone (240 ml, 4 rel vol), and dried in vacuum at 40° C. until the total amount of solvents were shown to be >1% (w/w) by thermogravimetric analysis. 58.3 g of (2R)-(3-amino-2-fluoropropyl)phosphinic acid form A was obtained after drying.

¹H-NMR (400 MHz, D₂O): δ 1.93 (1H, m), 2.13 (1H, m), 3.31 (2H, m), 5.14 (1H, dm, J=50 Hz), 7.07 (1H, d, J=528 Hz).

### Example 5

### Process Example 1

### Formation of (2R)-(3-amino-2-fluoropropyl)phosphinic acid form A

To a slurry of 450 kg (258.2 g/mole, 1.74 kmoles; weight at 100% assay) (2R)-3-[(tert-butoxycarbonyl)amino]-2-fluoropropylphosphinic acid ammonium salt in methanol (889 kg, 1125 L, 2.5 rel. vol.), concentrated sulphuric acid (230 kg, 2251 moles, 1.3 eqv.) was added over a period of 60 minutes while keeping Tᵢ=55±3° C. After the addition, the reaction mixture was heated to 62±3° C. and kept for 25 hours. After complete conversion the solution was cooled to 30±5° C., whereupon approximately 1 rel. vol. (355 kg, 450 L) was distilled off under reduced pressure (Tⱼ=60° C.). Methanol (357 kg, 450 L, 1 rel. vol.) is was added to the reaction slurry.

pH was adjusted to 5-6.2 by an addition of ammonium acetate (228 kg, 1.7 eqv.) dissolved in methanol (533 kg, 675 L, 1.5 rel. vol.). The addition is exotherm, why it is added over at least 30 minutes. After the additon the slurry was stirred for 30 minutes, precipitated salts were filtered off and the resulting filter cake was washed with methanol (533 kg, 675 L, 1.5 rel. vol.). The filtration must be performed within 3 hours to avoid precipitation of the title compound and a loss in yield as a result.

The filtered solution was concentrated to 650 L (1.4 rel. vol.) under reduced pressure (Tⱼ=60° C.). The resulting oil was temperature adjusted to 50° C. and seeded with crystals of the product from Example 3 (0.74 kg, 0.003 eqv.), whereupon ethanol (1067 kg, 1350 L, 3 rel. vol.) was charged over a period of 30 minutes. The slurry was aged for 1 hour, whereupon ethyl acetate (1412 kg, 1587 L, 3.5 rel. vol.) was added over a period of 30 minutes. The slurry was stirred at 50° C. for 15 minutes and then cooled to 0° C. over a period of 5 hours. After 1 hour at 0° C. the precipitated product was isolated and washed with a mixture of ethanol (356 kg, 450 L, 1 rel. vol.) and ethyl acetate (405 kg. 450 L, 1 rel. vol.). The isolated product was dried under vacuum at Tᵢ=40° C. (246 kg, 87%).

¹H-NMR (400 MHz, D₂O (4.7 ppm)): δ 1.83 (1H, m), 2.04 (1H, m), 3.22 (2H, m), 5.04 (1H, dm, J=49 Hz), 6.97 (1H, d, J=528 Hz).

### Example 6

### Process Example 2

### Formation of (2R)-(3-amino-2-fluoropropyl)phosphinic acid form A

246.5 kg (141.07 g/moles, 1.51 kmoles (2R)-(3-amino-2-fluoropropyl)phosphinic acid (crude) was elutriated in isopropanol (779 kg, 986 L, 4 rel. vol.) at 50° C. for 1.5 hours. The product was isolated, washed with isopropanol (487 kg, 616 L, 2.5 rel. vol.) and dried under vacuum at Tⱼ=45° C. (215 kg, 97%).

¹H-NMR (400 MHz, D₂O (4.7 ppm)): δ 1.85 (1H, m), 2.05 (1H, m), 3.23 (2H, m), 5.06 (1H, dm, J=49 Hz), 6.99 (1H, d, J=528 Hz).

### Example 7

### Process Example 3

### Formation of (2R)-(3-amino-2-fluoropropyl)phosphinic acid form A

291 kg (141.07 g/moles, 2.06 kmoles, weight at 100% assay) of (2R)-(3-amino-2-fluoropropyl)phosphinic acid was dissolved in water (435 kg, 435 L, 1.5 rel. vol.) and methanol (573 kg, 725 L, 2.5 rel. vol.) at 35° C. After 25 minutes the solution was clear filtrated and the filter was washed with methanol (229 kg, 290 L, 1 rel. vol). The solution was cooled to 25° C., followed by an addition of acetone (1833 kg, 2320 L, 8 rel. vol.) over a period of 3.5 hours. After 5 hours at 25° C. the product was isolated and washed with acetone (916 kg, 1160 L, 4 rel. vol.). The isolated material was dried under vacuum at Tⱼ=40° C. (259 kg, 87%).

¹H-NMR (600 MHz, D₂O (4.7 ppm)): δ 1.87 (1H, m), 2.08 (1H, m), 3.25 (2H, m), 5.09 (1H, dm, J=49 Hz), 7.01 (1H, d, J=527 Hz).

The crystals were analysed by X-ray powder diffraction (XRPD), see FIG. 2. The diffractogram of form A shows the following d-values given in Ångström and relative intensities:

Form A

| | Relative |
|---|---|
| d-value (Å) | Intensity |
| 7.8 | vs |
| 7.6 | w |
| 5.8 | m |
| 4.44 | vw |
| 4.34 | vw |
| 4.18 | w |
| 4.04 | m |
| 3.91 | m |
| 3.88 | vs |
| 3.79 | w |
| 3.58 | vw |
| 3.54 | w |
| 3.43 | vw |
| 3.32 | m |
| 3.08 | vw |
| 2.96 | vw |
| 2.92 | m |
| 2.80 | w |
| 2.69 | vw |
| 2.59 | w |
| 2.57 | vw |
| 2.53 | vw |
| 2.48 | m |
| 2.46 | w |
| 2.41 | vw |
| 2.33 | vw |
| 2.28 | vw |

The relative intensities are presented by the following definitions.

| Definitions used | % Relative Intensity |
|---|---|
| vs (very strong): | 100-70 |
| s (strong): | 70-40 |
| m (medium): | 40-10 |
| w (weak): | 10-5 |
| vw (very weak): | <5 |

1. A compound (2R)-(3-amino-2-fluoropropyl)phosphinic acid in crystalline form.

2. The compound (2R)-(3-amino-2-fluoropropyl)phosphinic acid form B, characterized in providing an X-ray powder diffraction pattern exhibiting substantially the following peak with d-value: 2.87 Å.

3. The compound (2R)-(3-amino-2-fluoropropyl)phosphinic acid form B according to claim 2, characterized in providing an X-ray powder diffraction pattern exhibiting substantially the following peaks with d-values: 6.2 Å, 4.05 Å and 2.87 Å.

4. The compound (2R)-(3-amino-2-fluoropropyl)phosphinic acid form B according to claim 3, characterized in providing an X-ray powder diffraction pattern exhibiting is substantially the following peaks with d-values: 6.2 Å, 4.10 Å, 4.05 Å, 2.87 Å, 2.60 Å and 2.47 Å.

5. The compound (2R)-(3-amino-2-fluoropropyl)phosphinic acid form B according to claim 4, characterized in providing an X-ray powder diffraction pattern exhibiting substantially the following peaks with d-values: 6.2 Å, 4.10 Å, 4.05 Å, 3.88 Å, 3.36 Å, 3.28 Å, 2.87 Å, 2.60 Å, 2.47 Å and 2.25 Å.

6. The compound (2R)-(3-amino-2-fluoropropyl)phosphinic acid form B according to claim 1, characterized in providing an X-ray powder diffraction pattern essentially as shown in FIG. 1.

7. A compound as defined in any one of claims 1 to 6 for use in therapy.

8. A pharmaceutical formulation comprising a compound according to any one of claims 1-6 in admixture with at least one pharmaceutically acceptable excipient.

9. Use of a compound according to any one of claims 1 to 6 as active ingredient in the manufacture of a medicament for the prevention or treatment of asthma, such as reflux-related asthma, pulmonary diseases, cough, laryngitis, chronic laryngitis, pain, failure to thrive, gastric emptying disorders, irritable bowel syndrome (IBS), functional gastrointestinal disorder (FGD), inhibition of transient lower esophageal sphincter relaxations (TLESR), emesis, gastric motility disorders, prevention of regurgitation, belching, hiccups, functional dyspepsia, gastro-esophageal reflux disease (GERD), pharyngitis, sinusitis, otitis media, dental erosions, aspiration prophylaxis, Barett's esophageus or non-erosive reflux disease (NERD).

10. A method of treatment or prevention of asthma, such as reflux-related asthma, pulmonary diseases, cough, laryngitis, chronic laryngitis, pain, failure to thrive, gastric emptying disorders, irritable bowel syndrome (IBS), functional gastrointestinal is disorder (FGD), inhibition of transient lower esophageal sphincter relaxations (TLESR), emesis, gastric motility disorders, prevention of regurgitation, belching, hiccups, functional dyspepsia, gastro-esophageal reflux disease (GERD), pharyngitis, sinusitis, otitis media, dental erosions, aspiration prophylaxis, Barett's esophageus or non-erosive reflux disease (NERD) which comprises administration of a therapeutically effective amount of a compound according to any one of claims 1 to 6, to a patient in need thereof.

11. A process for preparing (2R)-(3-amino-2-fluoropropyl)phosphinic acid form B as defined in any of claims 2 to 6, comprising the sequential steps of:
a. dissolving (2R)-(3-amino-2-fluoropropyl)phosphinic acid form A in a polar solvent;
b. adding an anti-solvent or a mixture thereof over a period of time and optionally cooling and optionally isolating the product; and
c. stirring at an elevated temperature for a period of time.

12. The process according to claim 11, wherein said polar solvent is methanol or water, or a mixture thereof.

13. The process according to any one of claims 11 or 12, wherein said anti-solvent is acetonitrile, acetone, ethanol, isopropanol or ethyl acetate.

14. The process according to any one of claims 11 to 13, wherein said period of time in step b is 10 hours.

15. The process according to any one of claims 11 to 14, wherein said temperature in step c is 40° C.

16. The process according to any one of claims 10 to 15, wherein said period of time in is step c is 33 hours.

17. A process for preparing (2R)-(3-amino-2-fluoropropyl)phosphinic acid form B as defined in any of claims 2 to 6, comprising the step of treating (2R)-(3-amino-2-fluoropropyl)phosphinic acid form A in a water-containing vapor phase optionally including vapor of other solvents a period of time, wherein the relative humidity is equal to or less than 55%.

18. The process according to claim 17, wherein (2R)-(3-amino-2-fluoropropyl)phosphinic acid form B is kept at saturated NaBr salt solution in water.

19. The process according to any one of claims 17 or 18, wherein the relative humidity is 51%.

20. The process according to any one of claims 17 to 19, wherein the temperature is 60° C.

21. The process according to any one of claims 17 to 20, wherein the period of time is 12 hours.

22. A process for preparing (2R)-(3-amino-2-fluoropropyl)phosphinic acid form B as defined in any of claims 2 to 6, comprising the step of keeping (2R)-(3-amino-2-fluoropropyl)phosphinic acid form A as a suspension in a solvent or a mixture of solvents with a water activity equal to or less than 25%; and then isolating the product from the suspension.

23. The process according to claim 22, wherein said solvent is a mixture of ethanol (90%, vol./vol.) and water (10%, vol./vol.).

24. The process according to claim 22, wherein said solvent is a mixture of acetone (50%, vol./vol.), methanol (40%, vol./vol.) and water (10%, vol./vol.).

25. The process according to any one of claims 22 to 24, wherein the temperature is kept at between 5° C. and 70° C.

26. The process according to claim 25, wherein the temperature is kept at between 20° C. and 50° C.
